(19) United States
(12) Patent Application Publication
(10) Pub. No.: US 2011/0093998 A1
(43) Pub. Date: Apr. 28, 2011

(54) Protective Insect Barrier Hood, Kit and Method Therefor

(76) Inventor: John P. Brennan, Minesing (CA)

(21) Appl. No.: 12/911,308

(22) Filed: Oct. 25, 2010

Related U.S. Application Data
(60) Provisional application No. 61/272,719, filed on Oct. 26, 2009.

(51) Int. Cl. A42B 1/00; A61F 9/00

(52) U.S. Cl. 2/4; 2/15

(57) ABSTRACT

Disclosed herein is a protective barrier insect hood which has a substantially rigid visor viewing portion connected thereto within an enlarged opening. The visor viewing portion is coupled to a supporting portion which supports the visor away from the user's face and also substantially maintains the hood away from the user's face. The supporting portion receives therein a portion of the user's cranial region. Also provided herein are methods for retro-fitting or installing a substantially rigid visor coupled to a supporting portion into a protective barrier insect hood and kit of parts for the same.

### CROSS-REFERENCE TO RELATED APPLICATION

The present application is related to and claims under 37 U.S.C. §119(e) benefit from and priority to U.S. Provisional Patent Application No. 61/272,719, filed Oct. 26, 2009, entitled “PROTECTIVE INSECT BARRIER HOOD, KIT AND METHOD THEREFORE”, the entire subject matter of which is hereby fully incorporated herein by reference.

### FIELD OF THE INVENTION

The present invention relates to protective barrier head coverings.

### BACKGROUND OF THE INVENTION

Various insects, such as mosquitoes, flies, bees and other flying pests are known to bite humans. The insects can be annoying to humans whom are required to work outdoors as well as those individuals enjoying leisure time. However, more importantly, insects are also known to carry and transmit diseases such as Malaria and West Nile Virus. Individuals are becoming more and more conscious of protecting themselves from biting and flying biting insects. Although numerous methods have been attempted to deter insect pests, such as chemical repellents which are convenient, they often have an unpleasant odor and are of limited value to a user since there is no physical barrier of protection afforded to the user. This may be of particular concern in areas where the flying, biting insect population is quite dense. Chemical repellents, furthermore, present the concern of allergic reactions by the user to the actual repellent. Therefore, physical barriers are a preferred method of protecting oneself against insect bites such as those from mosquitoes and flies.

However, although physical barriers for protecting one's self from flying, biting insects are preferred in various applications, there are significant draw-backs with current state of the art. For example, many examples of protective insect barrier hoods lack comfort as well as requiring the user to look through the protective mesh or fabric, thereby significantly decreasing the visual acuity of the user; which may unto itself present a significant risk.

### SUMMARY OF THE GENERAL INVENTIVE CONCEPT

At least one of the needs and objectives that will become apparent from the following description is achieved in an exemplary embodiment which comprises a protective barrier head covering. The protective barrier head covering comprises a hood portion formed of a barrier cloth and a substantially transparent face covering portion through which a user can see. The face covering portion includes a substantially rigid visor viewing portion which has an outer periphery with upper, lower and side boundaries and a supporting portion which is coupled near the upper boundary. The supporting portion is adapted such that it receives at least a portion of the user's cranial region. With regards to the hood portion, it is open at one end for receiving therethrough the user's head and neck regions. The hood portion includes an enlarged opening located near an upper and frontal portion of the hood portion which has a substantially complementary perimeter shape to the visor outer periphery. The visor is aligned within the enlarged opening and affixed to the hood portion about the outer periphery.

In some exemplary embodiments, the barrier cloth includes a mesh constituent. In various other exemplary embodiments, the barrier cloth includes a substantially non-permeable membrane constituent.

In some exemplary embodiments, the visor portion is formed from a substantially transparent material and may be tinted for limiting light transmission. Furthermore, in various exemplary embodiments, the visor may also include a magnifying region therein. In some exemplary embodiments, the visor portion includes at least one access port region. In still another exemplary embodiment, the access port region includes a complementary port flap portion for reversibly sealing the port region.

In some exemplary embodiments, the visor extends longitudinally from near a brow line to near an upper lip of the user's face and latitudinally from near a side edge of the user's face to near an opposing side of the user's face.

In some exemplary embodiments, the supporting portion is provided as a band for encircling the user's cranial region and supporting the visor.

In some exemplary embodiments, the supporting portion is provided as a pair of arms, each arm for frictionally engaging the user's cranial region near a temporal region thereof.

In some exemplary embodiments, the outer periphery of the visor is slightly larger than an outer periphery of the enlarged opening.

In some exemplary embodiments, the visor is affixed within the enlarged opening by way of double-sided tape. The double-side tape has a first side and a second side and the first side is affixed substantially continuously near the outer periphery of the visor and the second side is substantially continuously affixed near the outer periphery of the enlarged opening.

In some exemplary embodiments, the visor is affixed within the enlarged opening by way of single-sided tape. The single-sided substantially continuously bridges near the outer periphery of the visor and the near the outer periphery of the enlarged opening.

In some exemplary embodiments, the visor has an endless loop run channel formed therein located near the outer periphery for receiving a resilient bead and a portion of the barrier cloth. The barrier cloth may be fit within the run channel about the outer periphery of the enlarged opening and frictionally maintained in place by the resilient bead.

In some exemplary embodiments, the visor includes an endless loop run channel formed therein and located interior of the double-sided tape wherein the run channel is provided as a guiding formation for trimming excess barrier cloth from the visor viewing portion.

In another exemplary embodiment, there is provided a kit of parts for installing a substantially rigid visor viewing portion in a protective insect head covering. The kit comprises at least a hood portion formed of a barrier cloth which includes an open end for receiving therethrough a user's head and neck regions, a face covering portion including a substantially rigid visor viewing portion which has an outer periphery with upper, lower and side boundaries, a supporting portion coupled to the visor for supporting the visor and receiving at least a portion of the user's cranial region, at least one coupler for reversibly coupling the visor to an upper frontal portion of the hood portion, and substantially permanently or semi-permanently affixing means for substantially permanently affixing the visor near the outer periphery within the enlarged opening.

In some exemplary embodiments, the kit further comprises a cutter for cutting the enlarged opening substantially complementary in perimeter shape to the visor outer periphery and for receiving therein the enlarged opening the visor near the outer periphery. The cutter may be provided as a blade, scissors, a knife, a milk cutter, an Exacto™ knife or the like.

In some exemplary embodiments, the coupler is provided as a clip, a clothes pin, Velcro™ or the like, or a bobby pin.

In some exemplary embodiments, the affixing means may be provided as single-sided tape, double-sided tape or a ribbon for stitching the visor near the outer periphery to near a continuous edge of the enlarged opening.

In some exemplary embodiments, visor includes an endless loop run channel located near the outer periphery provided as a guiding formation for trimming excess barrier cloth or for receiving therein a resilient bead for securing the visor within the enlarged opening.

In some exemplary embodiments, the visor is sized such that it extends longitudinally from near a brow line to an upper lip of the user's face and latitudinally from near a side edge of the user's face to near an opposing side of the user's face.

In some exemplary embodiments, the supporting portion is provided as a band for encircling the user's cranial region and supporting the visor. In another exemplary embodiment, the supporting portion is provided as a pair of arms, each arm for frictionally engaging the user's cranial region near a temporal region thereof.

In another exemplary embodiment, there is provided a method of retro-fitting a face covering portion into a protective insect head covering. The method comprises at least the steps of:
a) providing a hood portion including an open end for receiving therethrough a wear's head and neck regions;
b) providing a face covering portion comprising a visor having an outer periphery and a supporting portion coupled near a top edge of the visor;
c) installing the face covering portion on a user's head, or a three-dimensional formation representative thereof, the face covering portion being supported on the cranial region of the user and oriented with the visor covering the user's face;
d) installing the hood portion over the user's head and covering the user's neck region;
e) reversibly coupling an upper and frontal portion of the hood portion to the visor using at least one suitable reversible coupler to maintain the visor and hood portion in a desired position;
f) removing the coupled hood portion and face covering portion from the user's head;
g) cutting an enlarged opening in the hood portion near the visor outer periphery;
h) affixing the visor within the enlarged opening; and
i) uncoupling the reversible coupler.

In some exemplary embodiments, the coupler may be provided as a clip, a clothes pin, Velcro™ or the like, a clip, or a bobby pin.

In some exemplary embodiments, cutter in the cutting step (g) may be provided by way of a blade, scissors, a knife, a milk cutter, an Exacto™ knife or the like.

In some exemplary embodiments, the visor is affixed within the enlarged opening in step (h) using single-sided tape, double-sided tape or a ribbon for stitching the visor near the outer periphery to near a continuous edge of the enlarged opening.

In some exemplary embodiments, the visor includes an endless loop run channel located near the outer periphery and step (h) further comprises placing an edge of the enlarged opening into the run channel and sandwiching the edge of the enlarged opening within the run channel using a friction-fit resilient bead also received into the run channel. Furthermore, the run channel in various other alternative embodiments may be used as a guiding formation for trimming excess cloth from the hood portion about the perimeter of the enlarged opening.

### BRIEF DESCRIPTION OF THE DRAWINGS

Several embodiments of the present invention will be provided, by way of examples only, with reference to the appended drawings, wherein:

FIG. 1 is a perspective view of an embodiment of the protective barrier hood mounted on a user's head;

FIG. 2A is a perspective view of an embodiment of a face covering portion;

FIG. 2B is a perspective view of an embodiment of the face covering portion;

FIG. 2C is a perspective view of an embodiment of the face covering portion coupled to the hood portion;

FIG. 2D is a perspective view of an embodiment of a hood portion;

FIG. 2E is a sectional view of an alternative embodiment of the face covering portion of the protective barrier hood of FIG. 1;

FIG. 3A is a perspective view of an embodiment of the protective barrier hood detailing the coupling of the visor and the hood portion;

FIG. 3B is a expanded perspective view of FIG. 3A of a single-sided tape coupling of the visor to the hood portion embodiment;

FIG. 3C is an expanded perspective view of FIG. 3A of a double-sided tape coupling of the visor to the hood portion embodiment;

FIG. 3D is an expanded perspective view of FIG. 3A of a run channel and resilient bead coupling of the visor to the hood portion embodiment;

FIG. 3E is an expanded perspective view of FIG. 3A of a ribbon stitching coupling of the visor to the hood portion embodiment;

FIG. 4A is a frontal view of an embodiment of the face covering portion supported on a user's cranial region;

FIG. 4B is a frontal view of an embodiment of the face covering portion supported on a user's head covered by the hood portion with reversible couplers attached;

FIG. 4C is a frontal view of an embodiment of the face covering portion supported on a user's head covered by the hood portion with reversible couplers attached;

FIG. 4D is a frontal view of an embodiment of the protective barrier hood the inner window removed; and

FIG. 5 is a perspective view of the visor viewing portion with a magnifying region.

### DESCRIPTION OF EXEMPLARY EMBODIMENTS

It should be understood that the invention is not limited in its application to the details of construction and the arrangement of components set forth in the following description or illustrated in the drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Furthermore, and as described in subsequent paragraphs, the specific mechanical, other configurations illustrated in the drawings are intended to exemplify embodiments of the invention. However, other alternative mechanical or other configurations are possible which are considered to be within the teachings of the instant disclosure.

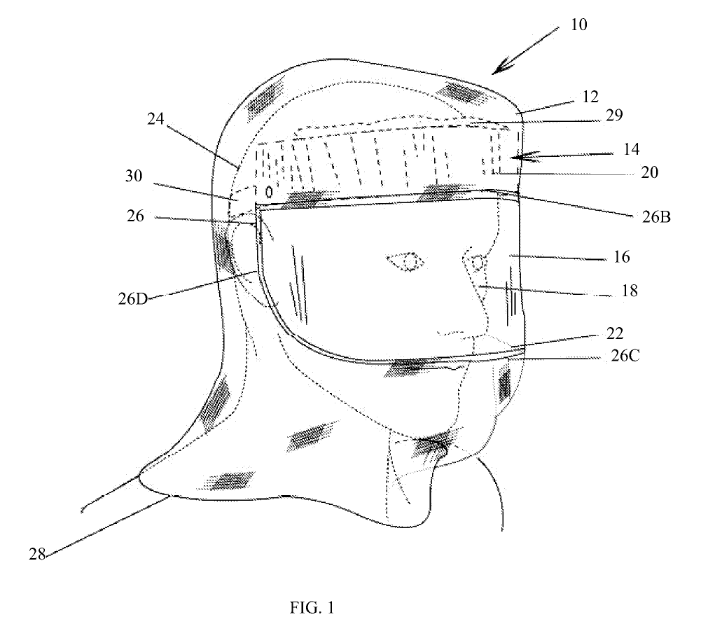

With reference to FIG. 1, a protective barrier head covering 10 is provided comprising a cloth hood portion 12 and having inset therein a face covering portion 14 through which the user can see. A substantially rigid visor viewing portion 16 is provided as part of the face covering portion 14, as is shown in FIG. 1 for protecting the facial region of a user 18 from insects and debris. During use, the face covering portion 14, including the transparent visor 16, is supported on a portion of a user's cranial region 24. The face covering portion 14, includes a supporting portion or upper visor frame 20 for receiving therein at least a portion of the user's cranial region 24. The supporting portion 20, as shown in FIG. 1, is coupled near a visor upper boundary 26B and fashioned such that the visor 16 is held away from the user's face. An enlarged opening 22 is provided near the frontal upper portion of the hood 12 for receiving the visor 16 of the face covering portion 14.

During use, the hood portion 12 of the head covering 10 is placed over the user's head via an open end 28 head such that the user's head is encased within the head covering 10. The supporting portion 20 receives a portion of the user's cranial region 24 and the visor 16 is oriented to shield the user's face as is shown in FIG. 1. The cloth of the hood then surrounds the remainder of the head, thus encasing the head of the user 18 and providing a barrier of protection to the user 18.

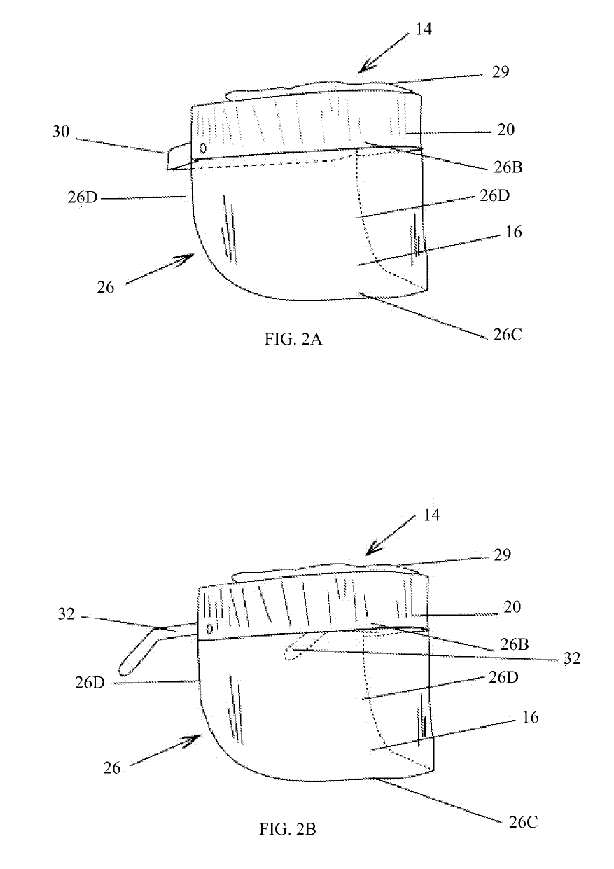

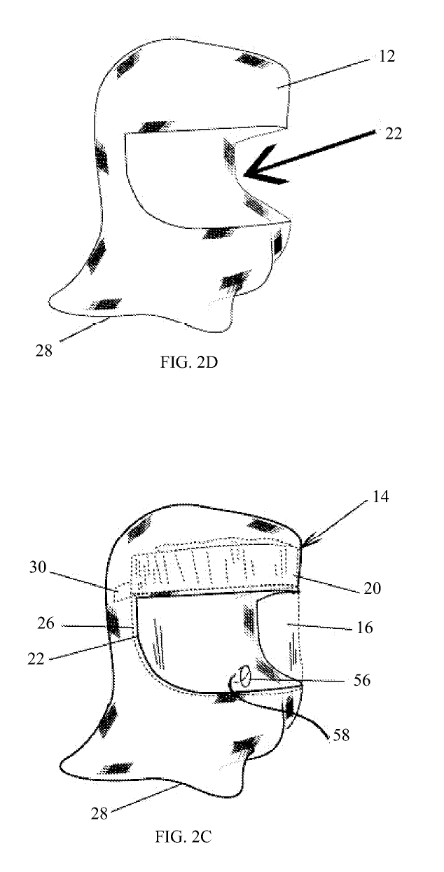

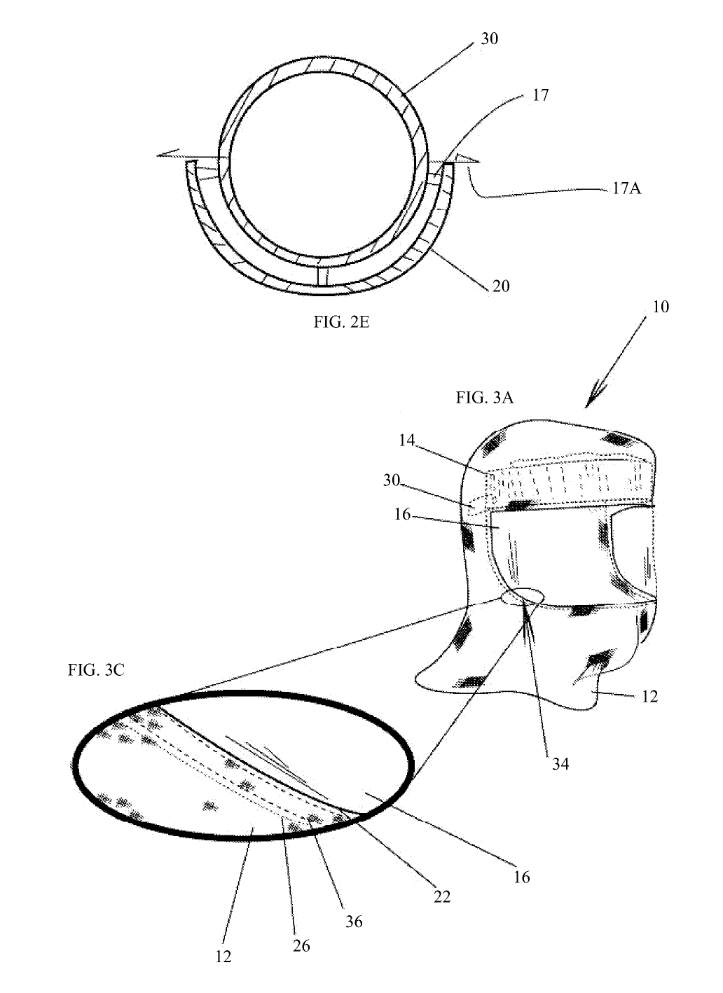

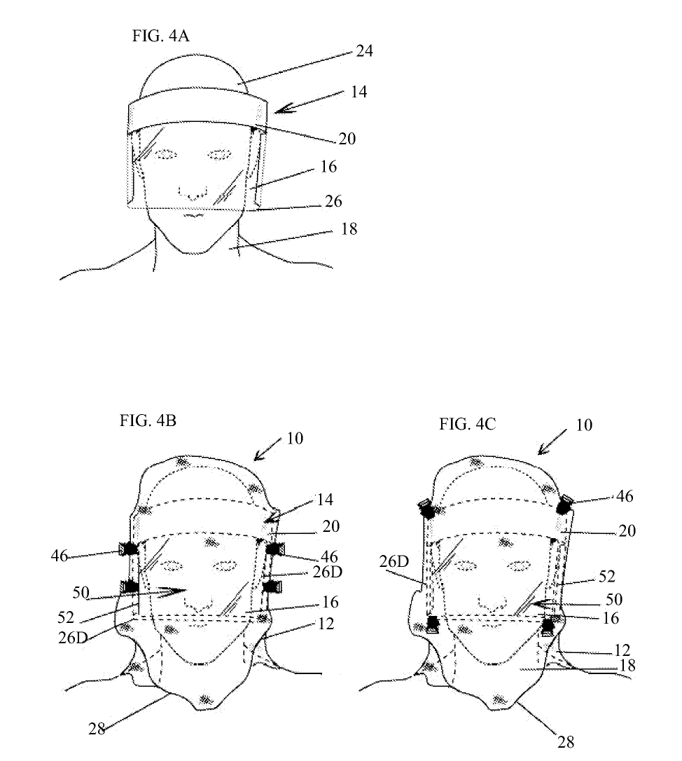

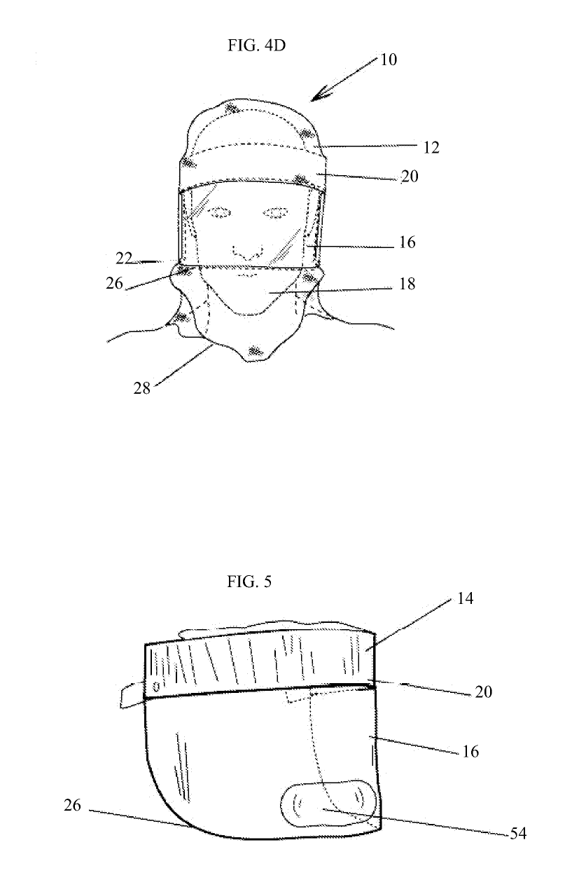

Regarding the construction and the interaction of the face covering portion 14, the visor 16 is coupled to the supporting portion 20 as is noted above and as shown in FIG. 2A. The visor 16 has an outer periphery 26; the outer periphery 26 having upper, lower and side boundaries, 26B, 26C and 26D respectively as is shown in the figures. The visor 16 is coupled near the upper boundary 26B to the supporting portion 20. Furthermore, as is shown in FIGS. 1 and 4A to 4D, the visor 16 extends longitudinally from near the user's brow line to near the user's upper lip and latitudinally from the near side edge of the user's face to near the opposing side of a user's face. Other dimensions of the visor 16 may also be utilized or desired.

With reference to FIG. 1, the supporting portion 20 is designed to keep the visor 16 separated from the user's facial region during use. The supporting portion 20 may be provided with a padding 29 to keep the visor 16 separated from the user's facial region as well as to increase user comfort. The padding in some exemplary embodiments may also include a single post or a plurality of posts 17 (as shown in FIG. 2E) operatively coupled to the supporting portion 20 in order to keep the visor 16 separated from the user's facial region. In other exemplary embodiments, the posts 17 may be included without the padding. Moreover, in some exemplary embodiments, the posts 17 may be adjustable as shown schematically by the arrow 17a in length for accommodating the use of separate eyewear with the device 10, for example sunglasses or corrective eyewear of a user. The adjustable posts 17 may enhance the mental comfort, for example alleviating to a degree claustrophobia which may be caused by the device 10 and/or aid in fog control on the visor 16. Furthermore, the supporting portion 20 may, in certain exemplary embodiments, be provided with an adjustable band 30 which receives a portion of the user's cranial region 24 as shown in FIGS. 1, 2A, 2C and 3A. For example, the adjustable band 30, coupled to the supporting portion 20, may be provided such that the head covering 10 may accommodate different users, each having different cranial circumferences. In various other embodiments, the supporting portion 20 may comprise a pair of arms 32 (FIG. 2B). The arms 32 are provided to reversibly frictionally secure the supporting portion 20 to the user's cranial region 24. Furthermore, the arms 32 may be designed to engage a user's ears.

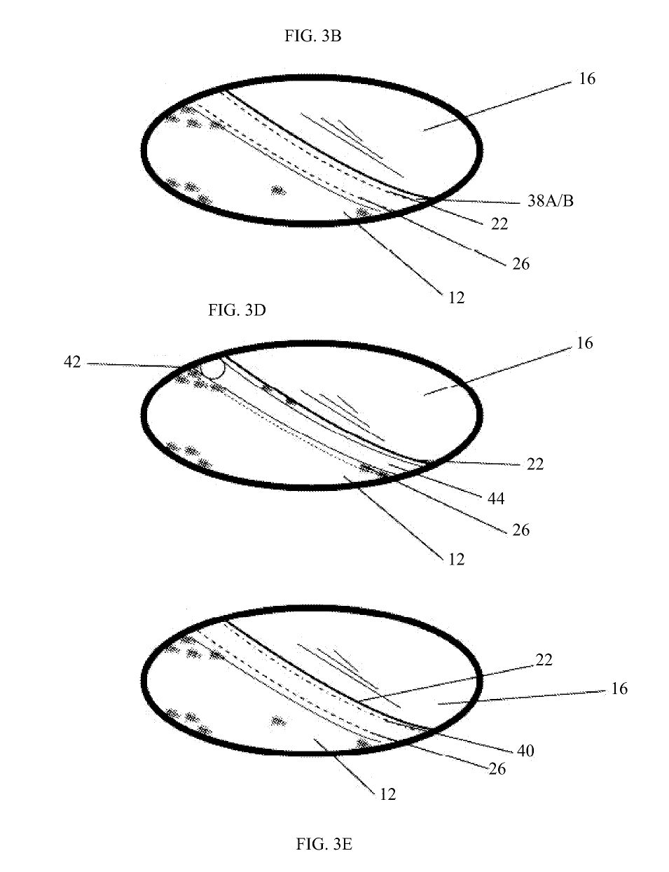

With reference to FIG. 2D, the hood portion 12 includes an enlarged opening 22. The enlarged opening 22 is fashioned to have a substantially complementary shape to the outer periphery 26 of the visor 16 as is shown in FIG. 2C. However the enlarged opening 22, located in the frontal and upper portion of the hood 12, is marginally smaller than that of the visor outer periphery 26 in order that the visor 16 can be coupled substantially continuously to the hood 12 near the periphery of the enlarged opening 22. Therefore, the visor 16, and thus the face covering portion 14, can be affixed to the hood portion 12 by way of suitable affixing means 34 as is shown in FIG. 3A and as in exemplary embodiments in FIGS. 3B to 3E. In certain exemplary embodiments, for example, double-sided tape 36, as shown in FIG. 3C may be utilized as suitable affixing means 34. Using double-sided tape 36, for example, one side of the double-sided tape 36, the first side, is affixed in a substantially continuous loop near the outer periphery 26. The other side of the double-sided tape 36, the second side, is then affixed complementarily to the hood portion 12 near the periphery of the enlarged opening 22. In another exemplary embodiment, shown in FIG. 3B, the visor 16 may be affixed near the periphery of the enlarged opening 22 by way of, for example, single-sided tape 38a bridging from near the visor outer periphery 26 to near the periphery of the enlarged opening 22 in a substantially continuous loop either interior the hood or exterior the hood. Furthermore, a second substantially continuous loop of single-sided tape 38B may be placed either interior the hood, as may be the case if the aforementioned substantially continuous loop of single-sided tape 38A is placed exterior the hood and vice versa, bridging near the visor outer periphery 26 and near the periphery of the enlarged opening 22 (not shown). Therefore, near the periphery of the enlarged opening 22 and near the visor outer periphery 26 may be bridged and, in effect sandwiched, between two layers of single-sided tape 38A and 38B, thus maintaining and affixing the visor within the enlarged opening 22. In another exemplary embodiment, as shown in FIG. 3E, the visor 16 may be affixed and maintained within the enlarged opening 22 by way of, for example, stitching 40. In this embodiment, a ribbon of stitching 40 is placed in a substantially continuous loop near the periphery of the enlarged opening 22 and depends through the visor 16 near the outer periphery 26, thereby maintaining the visor within the enlarged opening 22. In a further exemplary embodiment, the visor 16 may include a continuous loop run-channel 42 located near the outer periphery 26 as is shown in FIG. 3D. In this embodiment, the visor 16 is maintained within the enlarged opening 22 by way of the hood portion 12, near the periphery of the enlarged opening 22, being placed in the run-channel 42 and fictionally maintained within the run-channel by way of a substantially continuous resilient bead 44 being frictionally fit also in the run-channel 42. The periphery of the enlarged opening 22 is therefore sandwiched within the run-channel between the bead 44 and the visor 16.

Now having provided a general discussion of the construction of the protective barrier head covering 10, a discussion of various materials suitable for various parts of the head covering 10 is provided.

The hood portion 12, being provided as a barrier cloth, may, in various embodiments be comprised of different types of fabrics. For example, the hood portion 12 in various embodiments may be comprised of a mesh or netting having suitable density such flying and biting insects cannot, substantially, penetrate through the cloth to the user 18. The mesh density is suitably selected to allow for breathability such that the comfort of the user 18 is maintained. In other exemplary embodiments, the barrier cloth may be provided as a substantially non-permeable membrane to provide a substantially non-permeable protective barrier head covering 10 to the user 18. Additionally, the hood portion 12 may, in some exemplary embodiments be provided as a bug jacket (not shown).

In some exemplary embodiments, the visor 16 may be formed of a suitable material such that protection to the user 18 is afforded, yet allowing the user 18 a substantially unobstructed viewing area. For example, suitable visor materials may be, among other materials, polymers, Polycarbonate, Polyvinylchloride, Fluoroplastics, Polyethylene, Polymethylpentene, Polystyrene, Styrene Acrylonitrile, Acrylonitrile Styeren Acrylate, theremoplastics, epoxies glass and transparent crystalline matrices. In various embodiments the visor 16 may also be tinted to limit light transmission, for example, for use in bright light conditions. Furthermore, in various exemplary embodiments, the visor 16 may also have incorporated therein a magnifying region 54 as is shown in FIG. 5. The magnifying region, since it is held a substantially constant distance from the user's face, by virtue of the supporting portion 20, may in certain applications be useful to the user 18. Additionally, in various exemplary embodiments, the visor 16 may have incorporated therein at least one access port region 56. The port region 56, as shown in FIG. 2C by way of example, may be located near the user's mouth so as to allow access for drinking using, for example, a straw. A port region 56 may, for example in various exemplary embodiments, also be located near the user's eye region (not shown) so as to allow the user unobstructed viewing access to secondary ocular devices (not shown) such as, for example, a camera, binoculars or a weaponry sight device. Furthermore, the port region 56 may have a complementary flap portion 58 for reversibly sealing the access port region 58 as shown in FIG. 2C.

Methods to retro-fit or install a face covering portion 14 into a hood portion 12 are also provided.

To form the device 10, the backing of one side of double-sided tape 36 is removed and the double-sided tape 36 is secured substantially continuously near an outer periphery 26 of the visor 16. The supporting portion 20, having a visor 16 coupled thereto is then positioned comfortably on a user's head about the cranial region 24 and adjusted such that the visor 16 is aligned substantially centrally on the user's face as is shown in FIG. 4A. In some instances a model of a head, for example a human bust (not shown) may be employed in place of a user's head in accordance with the exemplary method. With reference to FIG. 4B, once the face covering portion 14 is aligned in a desired position with the user's face, a hood portion 12, having an open end 28 for receiving therethrough a user's head and neck region, is draped over the user's head such that the visor 16 is inside the hood portion 12. The portion of the hood 12 covering the visor is then suitably reversibly coupled by way of at least one reversible coupler 46 to the visor 16 about at least one side of the outer periphery 26 boundaries as shown in FIG. 4B. The portion of the hood 12 in front of the visor 16, which is to become an inner window 50 (FIGS. 4B and 4C) in a subsequent step, is then made taught against the visor 16 and suitably reversibly coupled using at least another reversible coupler 46 about an opposing boundary of the outer periphery 26. For example, the portion of the hood 12 may be reversibly coupled to a side boundary 26D near the left side of the user's face, made taught, and then reversibly coupled to the opposing side boundary 26D near the right side of the user's face. In a subsequent step, the face covering portion 14, now reversibly coupled to the hood portion 12 is removed from the user's head being careful not to alter the position of the couplers 46 relative the hood portion 12 and the face covering portion 14. Once the reversibly coupled face covering portion 14 and hood portion 12 are removed from the user's head, the remaining backing is removed from double-sided tape 36 and affixed, either near the upper boundary 26B or the lower boundary 26C of the outer periphery, as in the case of the current exemplary embodiment where the couplers 46 are coupled near the side boundaries 26D. Of course, in cases where the couplers 46 are reversibly coupled near the upper and lower boundaries 26B and 26C, as is shown in FIG. 4C, the remaining backing of the double-sided tape 36 would be removed from near the side boundaries in this step. Once the backing is removed from the double-sided tape 36, a corresponding portion of the hood 12 is pressed into the tape to be affixed thereto. The opposing boundaries, in this case, for example, the upper boundary 26B and the lower boundary 26C are affixed to the hood 18 in the same manner. The couplers 46 are then disengaged from coupling the visor 16 and the hood portion 12 and the backing of the double-sided tape 36 underlying these boundaries is similarly removed and the portion of the hood 12 is pressed into the tape, thereby affixing the side boundaries 26D as is shown in FIG. 3C. Using a suitable cutter 48 (not shown), the portion of the hood 12 covering the visor 16 is then cut to remove the inner window 50 (FIGS. 4B and 4C), thus creating an enlarged opening 22 in the upper and frontal portion of the hood 12. The face covering portion 12 is now substantially permanently or semi-permanently affixed to the hood 12 and creates the protective head covering 10 as substantially described above and shown in the figures wherein the visor 16 creates a substantially rigid transparent viewing area for the user 18 during use as generally shown in FIG. 4D. As an optional step, thus, single-sided tape 38A may be used to bridge the hood portion 12 enlarged opening 22 to the visor 16 to further affix or provide a more finished look to the protective head covering 10.

In another example, a supporting portion 20, having a substantially rigid visor 16 viewing portion coupled thereto is positioned comfortably on a user's head. In some instances a model of a head, for example a human bust (not shown) may be employed in place of a user's head in accordance with the exemplary method. The face covering portion is positioned about the cranial region 24 of the user's head and adjusted such that the visor 16 is aligned substantially centrally on the user's face as is shown in FIG. 4A. With reference to FIGS. 4B and 4C, once the face covering portion 14 is aligned in a desired position with the user's face, a hood portion 12, having an open end 28 for receiving therethrough a user's head and neck region, is draped over the user's head such that the visor 16 is inside the hood portion 12. The portion of the hood 12 covering the visor 16 is then suitably reversibly coupled by way of at least one reversible coupler 46 to the visor 16 about at least one side of the outer periphery 26 boundaries as shown in FIG. 4B. The portion of the hood 12 in front of the visor 16, which is to become an inner window 50 (FIGS. 4B and 4C) in a subsequent step, is then made taught against the visor and suitably reversibly coupled using at least another reversible coupler 46 about an opposing boundary of the outer periphery 26. For example, the portion of the hood 12 may be reversibly coupled to a side boundary 26D near the left side of the user's face, made taught, and then reversibly coupled to the opposing side boundary 26D near the right side of the user's face. In a subsequent step, the face covering portion 14, now reversibly coupled to the hood portion 12 is removed from the user's head; being careful not to alter the position of the couplers 46 relative the hood portion 12 and the face covering portion 14. Once the reversibly coupled face covering portion 14 and hood portion 12 are removed from the user's head, single-sided tape 38A is affixed to near an outer periphery 26 of the visor 16, along the boundaries 26B and 26C inside the hood 12 bridging to a portion of the hood 12. However, in a case where the visor 16 is reversible coupled to the hood portion 12 along the upper boundary 26B and the lower boundary 26C, the single-sided tape 38A would be first affixed to bridge the side boundaries 26D and the corresponding portions of the hood 12. The opposing boundaries, in this case, for example, the upper boundary 26B and the lower boundary 26C are affixed to the hood 18 in this manner. The couplers 46 are then disengaged from coupling the visor 16 and the hood portion 12 and additional single-sided tape 38A is used to bridge the visor 16 and the corresponding portions of the hood 12 along the side boundaries 26D interior to the hood 12, forming a substantially continuous loop of single-sided tape 38A bridging the visor and the portion of the hood 12 near the outer periphery 26 of the visor interior the hood 12. Using a suitable cutter 48, the portion of the hood 12 covering the visor 16 is then cut to remove the inner window 50 of the hood 12, thus creating an enlarged opening 22 as shown at 52 in FIGS. 4B and 4C, wherein the perimeter of the enlarged opening 22 is slightly smaller than the visor 16 outer periphery 26. A second substantially continuous loop of single-sided tape 38B is affixed to the visor 16 exterior the hood 12 and bridges the visor 16 to the enlarged opening 22 as shown in FIG. 3B. The face covering portion 14 is now substantially permanently or semi-permanently affixed to the hood 12 and creates the protective head covering 10 as substantially described above and shown in the figures wherein the visor 16 creates a substantially rigid transparent viewing area for the user 18 during use as generally shown FIG. 4D.

In another example a supporting portion 20, having a substantially rigid visor 16 viewing portion coupled thereto and a run-channel 42 incorporated in the visor 16 near an outer periphery 26 is positioned comfortably on a user's head. In some instances a model of a head, for example a human bust (not shown) may be employed in place of a user's head in accordance with the exemplary method. The face covering portion is positioned about the cranial region 24 of the user's head and adjusted such that the visor 16 is aligned substantially centrally on the user's face as is shown in FIG. 4A. With reference to FIGS. 4B and 4C, once the face covering portion 14 is aligned in a desired position with the user's face, a hood portion 12, having an open end 28 for receiving therethrough a user's head and neck region, is draped over the user's head such that the visor 16 is inside the hood portion 12. The portion of the hood 12 covering the visor 16 is then suitably reversibly coupled by way of at least one reversible coupler 46 to the visor 16 about at least one side of the outer periphery 26 boundaries as shown in FIG. 4c. The portion of the hood 12 in front of the visor 16, which is to become an inner window 50 (FIGS. 4B and 4C) in a subsequent step, is then made taught against the visor and suitably reversibly coupled using at least another reversible coupler 46 about an opposing boundary of the outer periphery 26. For example, the portion of the hood 12 may be reversibly coupled to a side boundary 26D near the left side of the user's face, made taught, and then reversibly coupled to the opposing side boundary 26D near the right side of the user's face. In a subsequent step, the face covering portion 14, now reversibly coupled to the hood portion 12 removed is from the user's head being careful not to alter the position of the couplers 46 relative the hood portion 12 and the face covering portion 14. Once the reversibly coupled face covering portion 14 and hood portion 12 are removed from the user's head, the inner window 50 is removed but cutting along a cut line 52 (FIGS. 4B and 4C) using a suitable cutter 48 (not shown) thus forming an enlarged opening 22 in the upper and frontal region of the hood 12. The cut line 52 is made such the inner window 50 is slightly smaller that the perimeter of the run-channel 42 as shown in FIG. 3D. The couplers 46 are then disengaged from coupling the visor 16 and the hood portion 12. A resilient bead 44 is subsequently frictionally-fit into the run channel 42 such that a portion of the hood 12 near the perimeter of the enlarged opening 22 is sandwiched within the run channel 42 between the visor 16 and the resilient bead 44 as is shown in FIG. 3D. The run channel 42, in various other exemplary embodiments, may also be used as a guiding formation trimming for excess hood 12 material with the cutter 48 to produce the enlarged opening 22 (not shown). Thus, as shown in FIG. 4D, the face covering portion 12 is now substantially permanently or semi-permanently affixed to the hood 12 and creates the protective head covering 10 as substantially described above and shown in the figures wherein the visor 16 creates a substantially rigid transparent viewing area for the user 18 during use.

In yet another example a supporting portion 20, having a substantially rigid visor 16 viewing portion coupled thereto is positioned comfortably on a user's head. In some instances a model of a head, for example a human bust (not shown) may be employed in place of a user's head in accordance with the exemplary method. The face covering portion is positioned about the cranial region 24 of the user's head and adjusted such that the visor 16 is aligned substantially centrally on the user's face as is shown in FIG. 4A. With reference to FIGS. 4B and 4C, once the face covering portion 14 is aligned in a desired position with the user's face, a hood portion 12, having an open end 28 for receiving therethrough a user's head and neck region, is draped over the user's head such that the visor 16 is inside the hood portion 12. The portion of the hood 12 covering the visor 16 is then suitably reversibly coupled by way of at least one reversible coupler 46 to the visor 16 about at least one side of the outer periphery 26, as shown in FIG. 4B. The portion of the hood 12 in front of the visor 16, which is to become an inner window 50 (FIGS. 4B and 4C) in a subsequent step, is then made taught against the visor and suitably reversibly coupled using at least another reversible coupler 46 about an opposing boundary of the outer periphery 26. For example, the portion of the hood 12 may be reversibly coupled to a side boundary 26D near the left side of the user's face, made taught, and then reversibly coupled to the opposing side boundary 26D near the right side of the user's face. In a subsequent step, the face covering portion 14, now reversibly coupled to the hood portion 12 removed is from the user's head; being careful not to alter the position of the couplers 46 relative the hood portion 12 and the face covering portion 14. Once the reversibly coupled face covering portion 14 and hood portion 12 are removed from the user's head, the inner window 50 (FIGS. 4B and 4C) is removed by cutting along a cut line 52, using a suitable cutter 48 (not shown), thus forming an enlarged opening 22 in the hood 12. The cut line 52 is made such that the inner window 50 is slightly smaller than the outer periphery 22 of the visor 16. The couplers 46 are then disengaged from coupling the visor 16 and the hood portion 12. In this exemplary embodiment, the visor 16 and the hood 12 are then stitched together using a suitable ribbon 40 (FIG. 3E) to form a substantially continuous loop thus adjoining the visor 16 and the hood 12 near the outer periphery 26 and the perimeter of the enlarged opening 22. The face covering portion 14 is now affixed to the hood 12, thereby creating the protective head covering 10 as substantially described above and shown in the figures wherein the visor 16 provides a substantially rigid transparent viewing area for the user 18 during use as is generally shown in FIG. 4D.

Furthermore, various features noted in exemplary methods of retro-fitting or installing a face covering portion 14 into a hood portion 12 to produce a protective insect barrier hood 10 may also have various other possible uses within the spirit of the current description. Additionally, certain features noted above may also have possible suitable variants and substitutions within the spirit of the invention.

Although not shown in the figures, the run channel 42 as described above may also be used as a guiding formation for trimming hood 12 material or for trimming excess hood 12 material following installation of the visor 16, with the cutter 48 to produce the enlarged opening 22. The guiding formation 42 may also be used to refine the enlarged opening 22 once the hood 12 and the visor 16 are adjoined. Once the enlarged opening 22 is produced, the hood 12 and the visor 16 may be affixed as described above with respect to the exemplary method embodiments employing double-sided tape 36, single-sided tape 38, stitching 40 and any combination thereof.

As noted above and shown in the figures, at least one coupler 46 is used to maintain a portion of the hood 12 in position for producing the inner window 50, and thus, the enlarged opening 22 in various exemplary embodiments. The coupler 46, for example, may be provided as any suitable means for reversibly adjoining the hood portion 12 and the visor 16 for the purposes of making the enlarged opening 22. By way of example, suitable couplers 46 may be provided as a clip, a clothes pin, Velcro™ or the like or a bobby pin.

As noted above, in various exemplary embodiments, a cutter 48 is used to produce the inner window 50, thereby creating the enlarged opening 22 in the hood 12 for retro-fitting a face covering portion 14 including a substantially rigid visor 16 into a protective insect head covering 10. The cutter 48 may be provided as any suitable means for producing the enlarged opening 22 along a cut line 52. For example, the cutter 48 may be provided as a blade, scissors, a knife, a milk cutter, an Exacto™ knife or the like.

In further exemplary embodiments, a kit of parts may be provided for use with a method of retro-fitting a face covering portion 14 into a hood portion 12 so as to produce a protective insect head covering 10 as described above. For example, the kit of parts may be provided with a face covering portion 14, at least one suitable coupler 46 and suitable affixing means 34, such as double-sided tape 36, single-sided tape 38, or a ribbon 40 for stitching the visor 16 within the enlarged opening 22. In another exemplary embodiment of the kit, visor 16 may be provided with a run-channel 42 formed continuously near the outer periphery 26. A resilient bead 44 may therefore be additionally provided for frictionally affixing the hood 12 to the visor 16 as substantially described above. Optionally, a cutter 48 such as, for example, a blade, scissors, a knife, a milk cutter, an Exacto™ knife or the like may also be provided for cutting the enlarged opening 22 in the hood 12. Furthermore, the coupler 46 may, for example, be provided as a clip, a clothes pin, Velcro™ or the like or a bobby pin.

In an exemplary embodiment, as shown in FIG. 1, the visor 16 may afford splash protection for the eyes, nose and mouth as well as insect protection to a user 18. Furthermore in various exemplary embodiments, although not shown in the figures, conventional eyewear such as for example, corrective eyewear, sunglasses and the like may be worn by the user in conjunction with the protective barrier insect hood 10. Additionally, the visor 16 placement away from the face may provide the benefit of air circulation in the user's facial region thus also aiding to reduce fog accumulation on the visor 16 from the user's breathing and perspiration. Improved insect protection about the facial region of the user 18 may also be observed in various exemplary embodiments since the hood portion 12 is not in contact with the user's face. In various other exemplary embodiments, the protective insect barrier hood 10 may be useful in certain applications aside from mere insect protection owing to the visor 16 affording a degree of protection to the user 18 from primary blood borne pathogens and other fluid borne contaminants or bodily fluids.

Also, since the visor 16 is coupled to the supporting portion 20 is mounted about the cranial region 24 of a user, the field of vision of the user 18 is substantially maintained as well as enhancing peripheral vision. The visor 16 is, thus, not prone to movement when the user 18 moves their head. The visor 16 therefore, moves substantially with the head of the user. Since the visor 16 is not prone to movement relative to the head of the user, the incorporation of a magnifying region 54 in the visor 16 may also be provided in various exemplary embodiments. For example, in such embodiments, the focal length between the eyes of the user and the magnifying region 54 remains substantially constant and may be useful in certain applications of various embodiments of the protective insect barrier hood 10.

Those of skill in the art will recognize certain modifications, permutations, additions and sub-combinations thereof of parts noted herein. While the protective head covering 10 and method of producing the invention have been described for what are presently considered the exemplary embodiments, the invention is not so limited. To the contrary, the invention is intended to cover various modifications and equivalent arrangements included within the spirit and scope of the appended claims. The scope of the following claims is to be accorded the broadest interpretation so as to encompass all such modifications and equivalent structures and functions.

What is claimed is:

1. A protective barrier head covering comprising:
a hood portion formed of a barrier cloth and a substantially transparent face covering portion through which a user can see;
the face covering portion including a substantially rigid visor viewing portion having an outer periphery with upper, lower and side boundaries and a supporting portion coupled near the upper boundary;
the supporting portion for receiving at least a portion of the user's cranial region;
the hood portion including an open end for receiving therethrough the user's head and neck regions;
the hood portion including an enlarged opening located near an upper and frontal portion of the hood portion substantially complementary in perimeter shape to the visor outer periphery; and
the visor being aligned within the enlarged opening and affixed to the hood portion about the outer periphery thereof.

2. A protective head covering as defined in claim 1, the visor extending longitudinally from near a brow line to near an upper lip of the user's face and latitudinally from near a side edge of the user's face to near an opposing side of the user's face.

3. A protective head covering as defined in claim 1, wherein the supporting portion is provided as a band for encircling the user's cranial region and supporting the visor.

4. A protective head covering as defined in claim 1, the supporting portion being provided as a pair of arms, each arm for frictionally engaging the user's cranial region near a temporal region thereof.

5. A protective head covering as defined in claim 1, wherein the outer periphery of the visor is slightly larger than an outer periphery of the enlarged opening.

6. A protective head covering as defined in claim 5, the visor being affixed within the enlarged opening by way of double-sided tape.

7. A protective head covering as defined in claim 6, the double-side tape having a first side and a second side;
the first side being affixed substantially continuously near the outer periphery of the visor;
the second side being substantially continuously affixed near the outer periphery of the enlarged opening.

8. A protective head covering as defined in claim 5, the visor having an endless loop run channel formed therein located near the outer periphery for receiving a resilient bead;
a portion of the barrier cloth about the outer periphery of the enlarged opening being frictionally fit within the run channel and maintained in place by the resilient bead.

9. A protective head covering as defined in claim 5, the visor being affixed within the enlarged opening by way of single-sided tape bridging from near the periphery of the visor to near the periphery of the enlarged opening.

10. A kit of parts for installing a substantially rigid visor viewing portion in a protective insect head covering, the kit comprising:
a hood portion formed of a barrier cloth;
the hood portion including an open end for receiving therethrough a user's head and neck regions;
a face covering portion including a substantially rigid visor viewing portion having an outer periphery with upper, lower and side boundaries;
the visor having a supporting portion coupled to thereto for supporting the visor and receiving at least a portion of the user's cranial region;
at least one coupler for reversibly coupling the visor to an upper frontal portion of the hood portion; and
substantially permanently or semi-permanently affixing means for substantially permanently affixing the visor near the outer periphery within the enlarged opening.

11. A kit as defined in claim 10, further comprising a cutter for cutting the enlarged opening substantially complementary in perimeter shape to the visor outer periphery and for receiving therein the enlarged opening the visor near the outer periphery.

12. A kit as defined in claim 10, the coupler being provided as a clip, a clothes pin, Velcro™ or the like, or a bobby pin.

13. A kit as defined in claim 10, the affixing means being provided as single-side tape, double-sided tape or a ribbon for stitching the visor near the outer periphery to near a continuous edge of the enlarged opening.

14. A kit as defined in claim 10, the visor including an endless loop run channel located near the outer periphery.

15. A kit as defined in claim 14, wherein the affixing means comprise the endless loop run channel near the outer periphery of the visor for frictionally receiving therein a resilient bead;
an edge of the enlarged opening is received into the run channel and maintained in place by being sandwiched within the run channel by frictional engagement of the resilient bead within the run channel.

16. A kit as defined in claim 10, the visor extending longitudinally from near a brow line to near an upper lip of the user's face and latitudinally from near a side edge of the user's face to near an opposing side of the user's face.

17. A kit as defined in claim 10, the supporting portion provided as band for encircling the user's cranial region and supporting the visor.

18. A kit as defined in claim 10, the supporting portion being provided as a pair of arms, each arm for frictionally engaging the user's cranial region near a temporal region thereof.

19. A method for retro-fitting a face covering portion into a protective insect head covering, the method comprising the steps of:
a) providing a hood portion including an open end for receiving therethrough a wear's head and neck regions;
b) providing a face covering portion comprising a visor having an outer periphery and a supporting portion coupled near a top edge of the visor;
c) installing the face covering portion on a user's head, or a three-dimensional formation representative thereof, the face covering portion being supported on the cranial region of the user and oriented with the visor covering the user's face;
d) installing the hood portion over the user's head and covering the user's neck region;
e) reversibly coupling an upper and frontal portion of the hood portion to the visor using a suitable reversible coupler to maintain the visor and hood portion in a desired position;
f) removing the coupled hood portion and face covering portion from the user's head;
g) cutting an enlarged opening in the hood portion near the visor outer periphery;
h) affixing the visor within the enlarged opening; and
i) uncoupling the reversible coupler.

20. A method as defined in claim 19, the visor being affixed within the enlarged opening in step (h) using single-sided tape, double-sided tape or a ribbon for stitching the visor near the outer periphery to near a continuous edge of the enlarged opening.

21. A method as defined in claim 19, step (h) further comprising placing an edge of the enlarged opening into an endless loop run channel located near the outer periphery and sandwiching the edge of the enlarged opening within the run channel using a friction-fit resilient bead;
the resilient bead also being received into the run channel.
